(19) United States
(12) Patent Application Publication
(10) Pub. No.: US 2006/0100567 A1
(43) Pub. Date: May 11, 2006

(54) Microsurgical tissue treatment system

(75) Inventors: Kevin S. Marchitto, Golden, CO (US); Stephen T. Flock, Arvada, CO (US)

Correspondence Address: Benjamin Aaron Adler, Ph.D., J.D.;Adler & Associates, 8011 Candle Lane, Houston, TX, 77071, US

(73) Assignee: Rocky Mountain Biosystems, Inc

(21) Appl. No.: 10/903,606

(22) Filed: Jul. 30, 2004

(51) Int. Cl. A61H 1/00; A61H 1/02; A61H 5/00; A61M 31/00

(52) U.S. Cl. 604/19; 604/500

(57) ABSTRACT

Provided herein is a device for altering a tissue in an individual comprising an applicator, a means to drive the applicator, and an abrasive material. The devices can further comprise a control means to monitor a physical property of the tissue. Also, provided herein is a device for ablating skin in an individual. Additionally, methods to use these devices are provided.

### CROSS-REFERENCE TO RELATED APPLICATIONS

This non-provisional application claims benefit of U.S. provisional No. 60/413,351, filed Sep. 25, 2002, now abandoned.

### BACKGROUND OF THE INVENTION

1. Field of the Invention

The present invention relates generally to the fields of biomedical engineering and drug delivery and tissue microsurgery. More specifically, the present invention provides a device and methods for treating tissues for the purposes of improving the permeation rates of substances across biological membranes and for achieving consistent results between treatment sites, as well as for tissue treatment for therapeutic or cosmetic reasons.

2. Description of the Related Art

Various methods have been used for facilitating the delivery of compounds across the skin and other membranes. In general, permeation of drugs through the skin occurs at a very slow rate, if at all. The primary rate-limiting step in this process is the passage of these compounds through the outermost layer of skin, called the stratum corneum. The stratum corneum is a very thin layer of dead cells that acts as an impermeable layer to matter on either side of this layer. The stratum corneum primarily provides the skin's barrier function. It has long been recognized that loss or alteration of the stratum corneum results in increased permeability to many substances; materials can more easily diffuse into or out of the skin. Several approaches have been used to ablate the stratum corneum for the purposes of drug delivery, however most fail to address the need for a high degree of precision and accuracy for consistent alterations of the stratum corneum and drug flux through the skin.

It has been demonstrated that electromagnetic energy induced alterations of the stratum corneum result in increased permeability to substances (see U.S. Pat. No. 6,424,863, U.S. Pat. No. 6,425,873, U.S. Pat. No. 6,315,722, U.S. Pat. No. 6,251,100, U.S. Pat. No. 6,056,738 and U.S. Pat. No. 5,643,252). Alternatively, compounds referred to as permeation enhancers, e.g., alcohol or drug carriers such as liposomes, can be used with some success to penetrate the stratum corneum. The barrier function of the skin presents a very significant problem to pharmaceutical manufacturers interested in topical administration of drugs, or in transcutaneous collection of bodily fluids.

U.S. Pat. No. 4,775,361 provides that electromagnetic energy produced by lasers may be used to ablate the stratum corneum in order to make the skin more permeable to pharmaceutical substances. Devices and methods for drug delivery using laser ablation systems have been described. U.S. Pat. No. 6,251,100 provides an improved method of administering a pharmaceutical composition, such as an anesthetic, through the skin of a patient without the use of a sharp or needle. This method includes the step of irradiating the stratum corneum of a region of the skin of the patient using a laser. By a selection of parameters, the laser irradiates the surface of the skin precisely to a selectable depth, without causing clinically relevant damage to healthy proximal tissue. A pharmaceutical composition is then applied to the region of irradiation. International Publication WO 00/57951 describes the use of non-ionizing energy, including lasers, to improve methods of administering pharmaceuticals in tissues, including the skin.

Devices and methods in U.S. Pat. No. 5,683,366, U.S. Pat. No. 5,697,536, U.S. Pat. No. 6,228,078, and U.S. Pat. No. 5,888,198 describe bipolar and monopolar RF electrosurgical devices that use a method of tissue disintegration as a means to ablate tissue prior to myocardial revascularization, tissue resurfacing or other surgical procedures. Radiofrequency energy has also been used to ablate tissues, and related methods have been used to enhance drug delivery through the skin. Publication WO 00/57951 describes methods and devices that use radiofrequency energy to improve permeation of substances across the stratum corneum. Certain applications describe feedback mechanisms that are used to prevent damage to viable tissue in the area surrounding the treatment site, such as described in U.S. Patent Publication No. 2002/0010414 A1 and WO 01/21068.

There are many procedures where a very precise ablation of tissue is of therapeutic benefit. For example, in ossicular bone surgery, the bones of the middle ear are sometimes removed, reshaped with a high-speed drill, and then reinserted in order to treat middle ear disease; the success of this procedure is very dependent upon surgical skill as the drill can remove large amounts of bone, if not used properly. In corneal sculpting or keratomileusis, tissue in the eye is reshaped with a laser in order to correct for vision disorders, such as myopia. These lasers are very expensive and require significant safety mechanisms because they employ potentially hazardous radiant energy. Dentists use several different tools to debride teeth of bacterial plaque and calculus, polish teeth and reshape teeth for aesthetic purposes. These tools can be inappropriate for very delicate and precise procedures and the result depends greatly on the skill of the dentist or oral hygienist.

A multitude of ablative procedures are performed on the visible surfaces of various tissues in order to improve their appearance, e.g., as in cosmetic tissue resurfacing treatments. Microdermabrasion is a common procedure where a thin layer of skin is removed with chemicals or a high-speed jet of crystals, whereupon small wrinkles or faults are smoothed out, as well as irregularities due to photodamage, acne scarring and scarring from surgical trauma. This process improves the appearance of the skin by giving it a smooth, fresh look. Conventional dermabrasion uses either a diamond fraise or a wire brush as a cutting tool powered by a handheld high-speed motor. The disadvantages of the powered tool include aerosolizing of infectious particles and blood splatter. Others have reported back-and-forth or circular motion manual use of abrading devices, including wire brushs or sandpaper.

A similar ablative process is also done, sometimes with lasers, for the purposes of burn debridement or scar revisions. Nail shaping and polishing also use an ablative process, although it is usually done manually by a trained person. Hair removal can be done several different ways, but the most popular for large areas of hair involve a lasers which ablates skin and sensitive parts of the hair follicle, however, such lasers are extremely expensive and require extensive training of the provider.

Recently, U.S. patent publication 200258902 described methods and devices for the ablation of barrier membranes using a shear device in order to enable sampling of biological fluids for diagnostic purposes and to enable delivering of active compounds for therapeutic purposes. That invention features a method for transporting a molecule through a mammalian barrier membrane following the ablation of the membrane with a shear device comprising a shear sheet containing at least one opening and a shear member, e.g., a shear blade such as those used in electric razors, where the sheet is contacted with the membrane such that a portion of the membrane is forced through the opening and the shear member as it moves parallel to the shear sheet, ablates the portion of the membrane exposed through the opening. The device further comprises a sensor, the feedback from which that modifies the driving force, e.g., by starting, speeding up, slowing down, or stopping the shear member's motion to enhance sufficient but not excessive membrane ablation.

For both drug delivery and biological fluid sampling, non-invasive and minimally invasive methods are preferred over invasive methods, such as needle injection, since they may easily be self-administered and are pain free. U.S. Pat. Nos. 5,250,028 and 5,843,113, PCT Patent Applications Nos. WO98/11937 and WO97/48440, and Henry et al. (Microfabricated Microneedles: A Novel Approach to Transdermal Drug Delivery, S. Henry, D. V. McAllister, M. G. Allen and M. R. Prausnitz, Journal of Pharmaceutical Sciences, Vol. 8, August 1998, pages 922-925), disclose perforation or disruption of the skin barrier membrane with mechanical means, e.g., with either small blades or needles, for such purposes. U.S. Pat. Nos. 5,421,816; 5,445,611 and 5,458,140 disclose, as a replacement for invasive sampling, the use of ultrasound to act as a pump for expressing interstitial fluid directly through visually intact, i.e., non-lanced, skin. Other means of treating a tissue to transiently increase the tissue permeability to enhance molecular transport for drug delivery and/or for sampling of interstitial fluids are disclosed in U.S. Pat. Nos. 5,019,034, 5,547,467, 5,667,491, 5,749,847, 5,885,211, and 5,441,490 and PCT Patent Application WO 95/12357.

It is notable that a consistent means of treatment are desirable. The Code of Federal Regulations (21 CFR 860.7(e)(1)) establishes that there is “reasonable assurance that a device is effective when it can be determined, based upon valid scientific evidence, that in a significant portion of the target population, the use of the device . . . will provide clinically significant results.” Devices which cannot be shown to provide consistent results between patients, or even within a single patient upon multiple use, will have minimal utility and may not be approvable for broad use.

Beyond devices it is generally desirable to develop medical products with critical controls that can deliver a precise result. Of critical concern is the delivery of many drugs. Certain drugs can be described as having a “broad” or “narrow” therapeutic index (TI). That is some drugs may be useful over a broad range of concentrations and thus are safe for the general population, while other drugs may only be effective over a narrow concentration range and may even be dangerous when administered in greater than recommended concentrations. This is particularly true where a drug has a narrow TI; the delivery of the drug must be controlled carefully so as to avoid potential harmful effects. The FDA (PMA Memorandum #P91-1: Clinical Utility and Premarket Approval) has established that devices which cannot be controlled may have limited utility. In particular, a drug delivery device may have limited utility if no assurance can be made that a consistent dosage is delivered throughout the patient population.

The drug-device combination must be capable of consistently delivering a dosage. As part of INDs and NDAs for administered drug products, bioavailability studies focus on determining the process by which a drug is released from the administered dosage form and moves to the site of action. Bioavailability data provide an estimate of the fraction of the drug absorbed, as well as its subsequent distribution and elimination. Bioavailability is defined in 21 CFR 320.1 as “the rate and extent to which the active ingredient or active moiety is absorbed from a drug product and becomes available at the site of action. For drug products that are not intended to be absorbed into the bloodstream, bioavailability may be assessed by measurements intended to reflect the rate and extent to which the active ingredient or active moiety becomes available at the site of action.” This definition focuses on the processes by which the active ingredients or moieties are released from a dosage form and move to the site of action. A delivery device which does not consistently release the same levels of a drug product due to the design of a product will have limited clinical utility, as there can be no assurance that a certain dosage has been delivered at any point in time.

Furthermore, studies to establish bioequivalence between two products are important to demonstrate safety and therapeutic efficacy in a product, and will be a benchmark for approval of drugs by regulatory bodies. Bioequivalence is defined at 21 CFR 320.1 as “the absence of a significant difference in the rate and extent to which the active ingredient or active moiety in pharmaceutical equivalents or pharmaceutical alternatives becomes available at the site of drug action when administered at the same molar dose under similar conditions in an appropriately designed study.”

As noted in the statutory definitions, both bioequivalence and product quality bioavailability focus on the release of a drug substance from a drug product and subsequent absorption into the systemic circulation. Where the test product generates variable effect at the site of action, as compared to those of the reference product, the product cannot be claimed as consistent, will not have great clinical utility and could be dangerous to use.

Control of delivery for current “patch” transdermal applications is achieved by delivering a fraction of what is “absorbable,” and either regulating the size of the dosage or the amount which is released from the vehicle. However, this simple means of regulation is not adequate for a system that could greatly accelerate the rate of percutaneous absorption. The condition of the skin and its hydration are significant factors in the percutaneous absorption of drugs. Some solubility of the substance in both lipid and water is thought to be essential. The aqueous solubility of a drug determines the concentration presented to the absorption site and the partition coefficient strongly influences the rate of absorption across the absorption site (Pharmaceutical Dosage Forms and Drug Delivery Systems, Ansel, H. C., Popovich, N. G. Allen, L. V. Eds., Williams & Wilkins, Baltimore, 1995). Vehicles that increase the hydration of the skin generally favor percutaneous absorption of drugs.

Whereas mechanisms are known in the art for protecting viable tissue surrounding the treatment site, the prior art is deficient in methods to achieve control over the alteration event in order to achieve variable rates of permeability. It would be beneficial for delivery devices to deliver consistently reliable dosages between sites and across a patient population or to assure that a consistent amount of material is collected from a site by adjusting the permeability characteristics of the treatment site itself, in addition to traditional methods in the formulation. Another benefit is recognized in the ability to regulate the depth of treatment as it relates to possible toxicity as well as flux, i.e., rate of permeation, and the surface area of the treatment site with relation to flux. Furthermore, it is desirable to attain simultaneous delivery of substances with minimal generation of heat.

Current cosmetic and therapeutic tissue treatments either involve a very expensive instrument, such as a laser, and highly trained care provider or the quality of the result depends greatly on the person doing the treatment. The inventors have recognized a need in the art for a device and methods that can alter tissue to produce a precise treatment with a high degree of control and that can be done economically and optionally at home by the untrained individual desirous of the treatment. The present invention further fulfills this long-standing need and desire in the art.

### SUMMARY OF THE INVENTION

The present invention is directed to a device for treating a tissue in an individual by altering or ablating the tissue comprising an applicator, a means to drive the applicator and an abrasive material.

The present invention also is directed to a device for ablating tissue of an individual comprising an applicator, a transducer to drive the applicator and an abrasive material comprised of particles of aluminum oxide having a particle size of about 30 microns to about 120 microns. The device may further comprise a lubricant of glycerol and water. The lubricant may be electrically conductive.

The present invention is directed further to a device for altering a tissue in an individual comprising an actuator, a transducer to drive the actuator, a controller to control the transducer, and a housing means. The housing comprises two wheels rotatably attached thereto. The tissue may be the stratum corneum.

The present invention is directed further to methods of altering tissue, ablating tissue, delivering pharmaceutical compounds, or collecting biomolecules from a tissue by using the devices disclosed herein.

The present invention is directed further still to methods of controlling permeability of a tissue using the devices disclosed herein having a feedback control means to monitor electrical properties, optical properties or thermal properties of the tissue.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1 depicts an actuator delivery system having an actuator encased in a housing to form a vibrating probe.

FIG. 2 depicts a cross-sectional view of the device of FIG. 1 when used on skin.

FIG. 3 depicts the surface of the actuator that is placed against the membrane to be treated displaying an array of chevrons on the inferior surface.

FIG. 4 depicts another embodiment of FIG. 1 where the probe and piezoelectric actuator are associated with at least one electrode that is in electrical contact with the ablation site of the membrane.

FIG. 5 depicts a horizontal displacement actuator.

### DETAILED DESCRIPTION OF THE INVENTION

In one embodiment of the present invention there is provided a device for treating a tissue in an individual comprising an applicator, a means to drive the applicator, and an abrasive material. The device may be contained within a patch or positioned on the end of a probe to be inserted into a body cavity. The tissue may be altered or at least a portion of the tissue may be ablated. The tissue may be a membrane such as the stratum corneum or a non-membranous tissue such as bone. In aspects of this embodiment the applicator may be a rough-textured surface disposed adjacent the tissue or may be an actuator in contact with the tissue.

Further in this embodiment the abrasive may be a biologically inert material. The abrasive may have particles ranging from about 30 microns to about 120 microns. A preferred range is from about 50 microns to about 90 microns. Representative examples of the abrasive are diamond, aluminum oxide, carborundum, or ice. In an aspect of this embodiment the abrasive may further comprise a lubricant. Examples of a lubricant are water, a hydrogel, a lipid, aqueous carbohydrate, petrolatum, or glycerol or a combination thereof.

In another aspect of this embodiment the driving means may be a piezoelectric material, a solenoid, a pressurized gas, an explosive discharge, a voice-coil, an electro- or magneto-responsive material, an electro- or magneto-rheologic material, a shape-memory alloy or polymer. An example of an electro- or magneto-responsive material is polypyrrol. An example of an electro-rheologic material is metallic filings dispersed in a viscous fluid and an example of a magneto-rheologic material is magnetic filings dispersed in a viscous fluid. A representative example of a shape-memory alloy is Nitonol. The driving means may further comprise an electrophoretic means, mechanical pressure, osmotic pressure, hydrostatic pressure or a diffusion gradient.

Yet another aspect of this embodiment provides a means to deliver a pharmaceutical. In one representative example the abrasive is the pharmaceutical or the abrasive comprises a lubricant containing the pharmaceutical. The pharmaceutical may be crystallized or powdered. In one example the crystals of pharmaceutical are frozen.

Another example of a delivery means is a reservoir having a permeable membrane. The reservoir contains the pharmaceutical which is controllably released through the permeable membrane. In both of these examples the pharmaceutical may be an anesthetic, nitroglycerin, an anti-nauseant, an antibiotic, a hormone, a steroidal antinflammatory agent, a non-steroid antiinflammatory agent, a chemotherapeutic agent, an anti-cancer agent, an immunogen, an anti-viral agent or an anti-fungal agent, or a diagnostic material. Representative examples of these types of pharmaceuticals are as disclosed infra.

In still yet another aspect of this embodiment the device may have a collection means to collect ablated tissue or a biomolecule after treating said tissue at a site of interest. An example of a collection means is a container operably connected to the device or an absorptive medium. Representative examples of an absorptive material are activated carbon, a dehydrated hydrogel or cotton.

Further to this embodiment there are provided control means to monitor feedback about a physical property of the tissue. The physical property may be an electrical property, an optical property or a thermal property. Representative examples of electrical properties are electrical impedence, electrical conductance, hydration, pH, or an endogenous electrical signal. An endogenous electrical signal may be one generated by a heartbeat or brain activity in an individual. Representative examples of optical properties are fluorescence or reflectance. Representative examples of thermal properties are thermal diffusivity or thermal conductivity.

In one aspect of feedback control, the control means to monitor an electrical property comprises at least one first active electrode in electrical contact at a site of interest on the tissue; a second return electrode in electrical contact distal to the first electrode at the site of interest; an optional electrically conductive fluid interface between the first and second electrodes and the site of interest on the tissue; and a controller to monitor an electrical current between the first electrode and the second electrode. The controller further has a microprocessor. In this aspect the first electrode(s) and the second electrode with an electrolyte in body fluid in the tissue comprise a galvanic cell.

In another aspect of feedback control, the control means to monitor an optical property comprises at least one source of radiant energy directed at a site of interest on the tissue; a light detector having optics with which to image the tissue thereon; and a controller to monitor the radiant energy source and the light detector and to analyze data received from the light detector, said controller further comprising a microprocessor.

In yet another aspect of feedback control, the control means to monitor a thermal property comprises at least one source of infrared energy directed at a site of interest on the tissue; an infrared detector having optics with which to measure infrared emission from said tissue thereon; and a controller to monitor the infrared energy source and the infrared detector and to analyze data received from the light detector, said controller further comprising a microprocessor.

Further to these feedback control aspects there are provided methods of controlling the permeability of a tissue in an individual comprising the steps of contacting a site of interest on the tissue with the device having one of the feedback control means as disclosed supra; treating the tissue to alter or ablate the tissue at the site of interest; monitoring an electrical, optical or thermal property of the tissue at the site of interest; applying an algorithm to evaluate the property monitored; comparing the value obtained for the monitored property to a predetermined value wherein the values correlate to the permeability of the tissue; and determining if the obtained value is at least equal to the predetermined value; and signaling the device via the controller to continue ablating or altering the tissue if the obtained value does not at least equal the predetermined value thereby controlling the permeability of the tissue at the site of interest.

In aspects of this embodiment the method may further comprise the step of delivering a pharmaceutical to the site of interest where the pharmaceutical is delivered during monitoring of the electrical, optical or thermal property or subsequent to reaching the predetermined value of this property. Further, a biomolecule may be collected through the altered or ablated tissue when the predetermined permeability value is reached. The predetermined value of the physical property may be a known value or be obtained prior to treating the tissue. The predetermined value may be obtained from the same individual or within a group of individuals.

In another embodiment of the present invention there is provided a device for ablating tissue of an individual comprising an applicator, a piezoelectric transducer to drive the applicator and an abrasive material comprised of particles of aluminum oxide having a particle size of about 30 microns to about 120 microns. Further to this embodiment the device may comprise a lubricant of glycerol and water. The lubricant may be electrically conductive.

In yet other embodiments of the present invention there are provided methods of treating tissue to alter and/or ablate the tissue at a site of interest in an individual by contacting the site of interest with the devices disclosed supra. In aspects of these embodiments the methods can comprise a further step of delivering a pharmaceutical to the site of interest. The pharmaceutical is delivered simultaneously with the altering or ablating step or subsequent thereto. In all aspects the pharmaceutical, the devices and tissues are as disclosed supra.

In related embodiments there are provided methods of collecting a biomolecule from a tissue by altering tissue or ablating tissue at a site of interest in an individual by contacting the site of interest with the devices disclosed supra and collecting the biomolecules through the altered tissue or through the ablated tissue. In all aspects the devices and tissues are as disclosed supra.

In still another embodiment of the present invention there is provided a device for ablating a tissue in an individual comprising an actuator, a transducer to drive the applicator, a controller to control the transducer and a housing means. The housing further comprises two wheels rotatably attached thereto. In this embodiment the actuator may be a piezoelectric actuator. The tissue to be ablated may be the stratum corneum. Further to this embodiment there is provided a method of ablating tissue comprising contacting the tissue in the individual at a site of interest with this device, applying downward pressure on the device to upwardly direct the tissue at the site of interest into the housing such that the tissue is in contact with the actuator; and ablating the tissue at the site of interest via the actuator.

The present invention provides a device for removal of thin layers of tissue and methods of use. The device comprises an abrasive member and a high frequency drive mechanism. The drive mechanism preferably is, but not limited to, a piezoelectric actuator. The actuator causes high frequency vibration in the plane defined by the tissue surface in at least one dimension relative to the site of treatment. Optionally, simultaneous motion in two or even three dimensions may be beneficial.

Furthermore, the drive mechanism may also include solenoids, high-pressure gas, explosive material, voice-coil, electro- or magneto-responsive materials, e.g., polypyrrol, electro- or magneto-rheologic materials, e.g., metallic or magnetic filings dispersed in a viscous fluid, or shape-memory alloys or polymers, e.g., Nitonol. The device may use an additional driving force to permeate substances into a site treated by the abrasive member. The driving force may optionally include, but not be limited to, electrophoretic means, mechanical pressure, diffusion gradients, osmotic pressure or hydrostatic pressure.

A safety interlock may be affixed to the device, or integrated into a patch, such that the device cannot be utilized unless the interlock is engaged, and only under proper use. For example, the interlock could be mechanical, electrical or optical. In the “on” position, either engaged or disengaged, the device may be operational. In the “off” position, the device would fail to be operational. This interlock prevents treating beyond a subscribed depth, and also prevents subsequent use of the same abrasive material on another patient.

A container may be attached to the distal end of the device such as to contain the abrasive and collect ablated tissue or other biomolecule. The container may be permanent or disposable. Alternatively, in a patch device, the container would be equivalent to a disposable or non-disposable component that is in contact with the skin. The container may be modified to hold, or receive through an opening, a pharmaceutical or other substance, which may then be delivered or collected simultaneously or shortly after ablating of the tissue occurs. The container may be integral to, or function independently of a safety interlock. Alternatively, an absorptive material may be used, e.g., activated carbon, dehydrated hydrogel or cotton.

A control means to monitor feedback about a physical property of the tissue may be used. The control means comprises monitoring various physical properties of tissue such as properties that can change when tissue is ablated or altered. For example, electrical impedance, electrical capacitance, pH, optical fluorescence or reflectance, either IR or visible, thermal diffusivity and conductivity, transepithelial water loss, ultrasonic reflection, or gaseous efflux may be measured.

In the case of electrical measurements to monitor the tissue, the device may also comprise an electrode or series of electrodes to measure electrical properties of the treatment site and provide feedback to the device. Current flow is used to modulate the oscillatory speed or extent of travel of the abrasive element. These electrical properties include, but are not limited to, electrical impedence or electrical capacitance. Once a desired measure of the electrical property is reached during treatment, feedback to the device may be used to control and monitor further treatment.

In general, the electrical impedence of the skin can approach values as high as 10⁸ ohms·cm². As successive layers of the stratum corneum are removed, this impedence can drop to a fraction of that value. This drop in impedence can be monitored as a measure of the degree of the process. Another aspect of the invention is that, with the other parameters set, the depth of treatment can be precisely controlled by continuously monitoring the impedence across the target area, and causing a feedback loop whereby the process is halted when a desired endpoint is met. Therefore, various settings on the device can be adjusted to allow successive reduction of the stratum corneum.

Control may be mediated through the creation of a galvanic cell between two monitoring electrodes and fluids encountered in the membrane as a result of treatment. The two monitoring electrodes in electrical contact with the treatment site and untreated site are composed of dissimilar metals. The tip of one electrode is placed adjacent the ablation site on tissue and the electrically conducting dissimilar metal plate of the other electrode is placed in contact with tissue at a location remote from the ablation site.

These electrodes and an electrolyte defined as body fluid present in the intervening tissue below the surface of the skin create a galvanic cell when the tip and plate have different work functions because of migration of electrical charges therebetween. That is when alteration or ablation at the treatment site occurs, charges generated by an electrochemical gradient between the electrodes begin to migrate. This migration of charges is increasingly efficient as the hydration level increases. Thus, the functionality of the galvanic cell may be monitored as a means to detect changes in hydration, and the information used to regulate the energy output of the device. For example, as the successive layers of stratum corneum are removed, the probes encounter a hydration gradient which results in increased conductance.

This last method may optionally require the probe to be in contact with the skin. Alternatively, contact with a liquid interface at the skin surface would minimize the effect of contaminants in the area that may have an electrically insulative effect. The information on conductance is then relayed to a controller which in turn adjusts the treatment of the target site to achieve a desired alteration or ablation. Alternatively, the control means consists of a means to measure the change in the charge storage characteristics of the skin, such that increasing “leakiness” to ions and/or charge, due to breakdown of the “skin battery” is an indication of the depth of treatment.

The device may also monitor endogenous impulses arising from the body by physiological processes, for example, electrical impulses generated by heart. Such impulses may include, but are not limited to, electrical impulses generated by heartbeats. The magnitude of these impulses increases with decreasing electrical resistance of the tissue being treated and so is a measure of the depth of treatment.

Optical properties of the tissue also may be monitored as a means of feedback control. When tissue is altered or ablated, it's optical properties change due either to molecular changes in the tissue itself or due to the exposure of underlying tissue with a different chemical makeup. For example, when the stratum corneum is removed from skin, the underlying epidermis fluoresceses strongly when exposed to the ultraviolet light of a Wood's lamp. When soft tissue is coagulated, it's scattering and absorption properties change and thus the reflectance changes also.

For use of optical measurements to monitor the tissue during alteration or ablation, the control means comprises at least one source of radiant energy, the output of which is directed at the tissue to be interrogated, a light detector with optics such that the interogated tissue is imaged onto the detector and a controller and microprocessor to modulate the radiant energy source, monitor the detector and to analyze the measurements. The controller further has a microprocessor.

When tissue is altered or ablated, the thermal properties of the tissue change due to molecular alterations in the tissue or due to exposure of underlying tissue of different properties. The properties of thermal diffusivity and thermal conductivity can be monitored by performing pulsed photothermal radiometry whereupon the tissue to be interrogated is heated slightly. For example, the radiant energy of an infrared diode laser directed at the tissue may be used.

The maximum temperature reached and the rate at which the tissue cools are a function of the thermal properties. The temperature of the tissue can be followed by measuring the infrared emission of the tissue with an infrared detector which is optically configured to image the tissue to be interrogated. In the case of skin, when the stratum corneum is completely ablated, a significant change in the infrared emissions from the skin occurs. This abrupt change can be used to controllably ablate the stratum corneum to a reproducible depth.

Depending on the tissue type, when tissue is altered or ablated, transepithelial water loss increases, if the tissue is skin or endothelial tissue, ultrasonic reflection due to changes in acoustic impedance occurs, and respiratory gases, i.e., oxygen and carbon-dioxide, diffuse out. Transepithelial water loss increases when the stratum corneum is altered or ablated because the barrier function of he stratum corneum is compromised and thus diffusion of molecules through the membrane is enhanced; this also explains enhanced gaseous efflux when skin is altered or ablated. Changes in tissue acoustic properties can occur upon alteration or ablation due, in part, to changes in the hydration of the tissue.

Measuring the change in the degree of hydration at the target site can control the depth of treatment, i.e., degree of hydration positively correlates with depth of treatment. Alternatively, by measuring the hydration level in a membrane before the application of a substance, the degree of hydration indicates the likely permeability of a substance through the treated site. The degree of hydration may be determined by corneometry or, preferably, by evaluation of conductance which becomes more efficient as increasing hydration is encountered. Further, the device can seek a pre-determined state of hydration, using this as a benchmark for standardizing permeability of a substance.

A feedback loop is created by a central controller which monitors information on hydration and uses an algorithm to compute relative or absolute hydration. The controller then signals the device to continue or cease the treatment process in order to seek the optimal depth of treatment with respect to hydration and permeability characteristics of a particular substance. The devices described herein are preferably used for alteration or ablation of a membrane, usually the stratum corneum of the skin. The device alters the stratum corneum in a manner that exposes increasingly hydrated layers of this skin layer, thereby increasing the percutaneous absorption of a substance through this layer. When an optimal threshold of hydration is reached the energy delivery is reduced or curtailed.

A desired effect can be obtained by varying the displacement of the piezoelectric actuator and the movement in more than one dimension. Broad surface area treatments may be obtained by applying the device to a treatment surface of greater surface area. The device is moved over the target site on the skin of the individual in order to obtain a large treated surface area. This allows for an efficacious drug dose to be delivered, but avoids local toxic effects due to too high a local concentration of the drug. A representative area of the target site is about 0.1 cm² to about 500 cm².

Additionally, the actuator may be moved in a second or third dimension through the addition of a second actuator, or other driving force, that provides movement in those dimensions. For example, an actuator that vibrates horizontally may be driven vertically, or in a rotating manner by a second driving force, which may, in turn, be an actuator. This movement in additional dimension(s) has the added benefit of providing a greater lubricating effect.

In general, the driving means may be a piezoelectric material, a solenoid, a pressurized gas or an explosive discharge, an electrolytic polymer, a magnetorheological material, an electrorheological material, e.g. dielectric gel of mixed with ERF between two flexible electrodes or lithium polymethacrylate, an electroresponsive metal, a shape-memory alloy, or a mechanical spring.

Grit or particle size of the abrasive material is a determining factor in the coarseness of the abrasion with greater particle size relating to greater ablation effect. An abrasive material with particles in the range of about 30 to about 120 microns is preferred, however larger or smaller particles are useful in some applications. The abrasive material may be diamond, aluminum oxide, carborundum which is preferably fixed to a pad driven by the drive mechanism or other material.

Particle size may be chosen to achieve a desired effect. For example, smaller particles may be used for polishing tissue or resurfacing skin for cosmetics or therapeutic purposes as in dermabrasion. Larger abrasive particles may be used to remove tissue. For example a moderate grit or particle of about 30 microns to about 90 microns is used in order to achieve a significant ablation effect while not tearing the tissue or abrading it in an irregular manner. Preferably, the device utilizes an abrasive material of approximately 50 to 90 microns that is driven by a piezoelectric actuator with displacement in the range of 50 to 500 microns. The action of the abrasive is to remove stratum corneum from the skin in order to improve the permeation of substances, such as drugs, into the skin, and the collection of fluids from the body.

Additionally, the abrasive material may be a pharmaceutical, such as lidocaine, in powdered or frozen crystallized form which acts as the abrasive as well as the drug that subsequently permeates the tissue. In powdered form the drug dissolves upon contact with moisture in the tissue; in the crystallized form the drug melts into a liquid form as a consequence of body heat.

Lubrication of the abrasive member may result from the presence of fluid in the sample, however the lubricating effect of fluid may diminish the abrasive effect of the particles. Nevertheless, an advantage can be derived in that application of a drug may take place simultaneously with the abrasion. Alternatively, a lubricant to irrigate and remove debris, as well as to provide a cooling effect, may be used. This irrigant may be applied as a cryogenic spray to provide an enhanced cooling effect.

Higher frequencies of oscillatory motion result in a more controllable lubricating effect. When particle size is controlled within the given parameters, high frequency treatment results in an effectual uniform surface for treatment, and prevents uncontrolled ablation. Frequencies greater than 1 kHz provide an adequate lubrication effect, however frequencies above approximately 20 kHz may generate too much heat, resulting in a tissue welding effect. A carefully controlled process using the aforementioned parameters results in successive removal of thin layers of tissue. These layers are approximately 1 to 5 micron in thickness. Thus, by controlling pulse number and duration, one may carefully control the depth of treatment at the micron level.

Other parameters which are controllable include the angle of incidence of the actuator with respect to the tissue. By increasing the angle, the ablation effect begins to extend into the membrane in a manner similar to an ultrasonic knife. For micromainpulation of tissue, it is desirable to probe with a fine-tipped actuator coated with the appropriate size abrasive particles. The longitudinal motion of the abrasive members, operating at a high frequency, thus become a lubricated saw.

High frequency vibration minimizes the pressure that must be applied to the surface, thus improving the control over the treatment as well as enabling the use of compact, lightweight applicators that can easily be affixed to the skin surface. In the case of surgical cutting, much less pressure need be applied, thereby minimizing the possibility of distortion of critical membranes or other structures. This “pressureless” surgical tool can provide clean, fast incisions with little or no undesirable damage to surrounding tissues.

One of the limitations of transcutaneous delivery of drug formulations is that the drug can be locally toxic at high doses, and therefore must be modulated to permeate the skin at a controlled rate. In the present case, modulation may occur by limiting the depth of the treatment, and by controlling the flux of the drug by delivering it over a larger surface area. Thus, a large surface area for the delivery of pharmaceutically active substances where those substances may adversely interact with tissues is provided for treatment. Further, substances that have poor permeability characteristics, even in the presence of an altered or ablated membrane, may be better delivered through a larger surface area.

The present invention provides a means for treating local pain or infections, or for application of a substance to a small specified area, directly, thus eliminating the need to provide high, potentially toxic amounts systemically through oral or intravenous administration. Locally acting pharmaceuticals such as alprostadil (for example, Caverject from Pharmacia & Upjohn), various antibiotics, antiviral or antifungal agents, or chemotherapy or anti-cancer agents, can be delivered using this method to treat regions proximal to the delivery site. Protein or DNA based-biopharmaceutical agents can also be delivered using this method.

Antigens derived from a virus, bacteria or other agent which stimulates an immune response can be administered through the skin for immunization purposes. The antigen is delivered through the outer layers of the stratum corneum, either singly or multiply, and the immunogen is provided in an appropriate formulation. For booster immunizations, where delivery over a period of time increases the immune response, the immunogen can be provided in a formulation that penetrates slowly through the treatment site, but at a rate faster than possible through unaltered skin.

Analgesics and other non-steroidal anti-inflammatory agents, as well as steroidal anti-inflammatory agents, may be caused to permeate through reduced stratum corneum to locally affect tissue within proximity of the irradiated site. For example, anti-inflammatory agents such as Indocin (Merck & Co.), a non-steroidal drug, are effective agents for treatment of rheumatoid arthritis when taken orally, yet sometimes debilitating gastrointestinal effects can occur. By administering such agents through laser-assisted perforation or alteration sites, these potentially dangerous gastrointestinal complications may be avoided. Further, high local concentrations of the agents may be achieved more readily near the site of irradiation as opposed to the systemic concentrations achieved when orally administered.

The substances used in this embodiment may be biological molecules such as pharmaceutical compounds. Representative examples of such substances are nitroglycerin, an anti-nauseant, a hormone, a steroidal antinflammatory agent, a non-steroid antiinflammatory agent, LHRH, a chemotherapeutic agent, an anti-cancer agent, an immunogen, an anti-viral agent or an anti-fungal agent. A representative example of an anti-nauseant is scopolamine. Representative examples of an antiobiotic are tetracycline, streptomycin, sulfa drugs, kanamycin, neomycin, penicillin, or chloramphenicol. Representative examples of a hormone is parathyroid hormone, growth hormone, gonadotropins, insulin, ACTH, somatostatin, prolactin, placental lactogen, melanocyte stimulating hormone, thyrotropin, parathyroid hormone, calcitonin, enkephalin, or angiotensin. Additionally, the substances of the present invention may be interstitial fluid or a diagnostic reagent. These substances may be removed from tissue using the methods disclosed herein.

The devices provided herein can be used to alter the stratum corneum to improve the collection of fluids, gases or other biomolecules through the skin. The fluid, gas or other biomolecule can be used for a wide variety of tests. A representative example of a use for interstitial fluid is to measure analytes. For example, the technique of the present invention may be used to improve the ability to sample extracellular fluid in order to quantify glucose or other analytes. Glucose is present in the extracellular fluid in the same concentration as or in a known proportion to the glucose level in blood.

The technique of successive removal of layers of dead or necrotic cells of the stratum corneum provides several advantages. Preferably, the stratum corneum is reduced, but not removed, so that its structural and biochemical makeup still permit drugs to permeate. Therefore, the skin after treatment still presents a barrier, albeit reduced, to external factors such as viruses and chemical toxins. Less energy is required for reduction than is required to entirely remove the stratum corneum, thus smaller and cheaper devices can be used. The technique also minimizes the damage to surrounding tissues providing a more rapid and efficient replacement of the stratum corneum.

As described herein, the invention provides a number of therapeutic and diagnostic advantages and uses. Embodiments of the present invention are better illustrated with reference to the Figure(s), however, such reference is not meant to limit the present invention in any fashion. The embodiments and variations described in detail herein are to be interpreted by the appended claims and equivalents thereof.

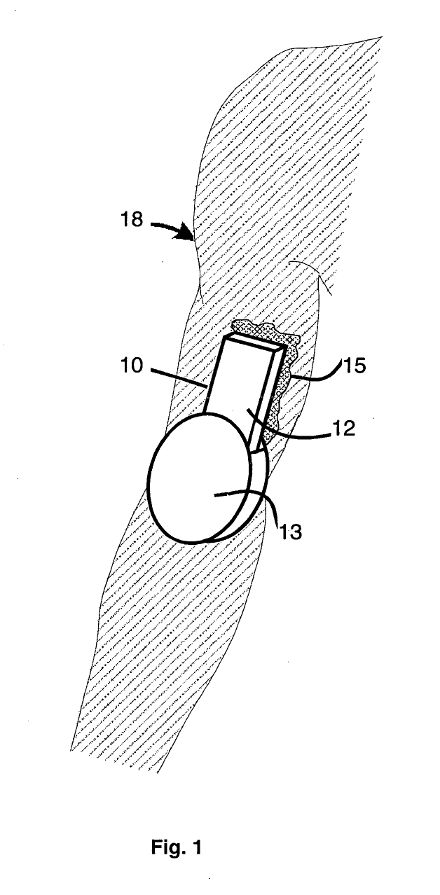

FIG. 1 depicts a device 10 which functions as a vibrating probe having a piezoelectric actuator 12 integrated into a housing 13. The actuator 12 delivers energy resulting in high frequency vibration which causes an ablation or alteration of the membrane 18. An abrasive 15 is applied between the actuator 12 and the membrane 18. Oscillatory movement of the actuator 12 in the plane defined by the membrane 18 causes ablation due to the repeated interaction of the abrasive 15 with the membrane 18.

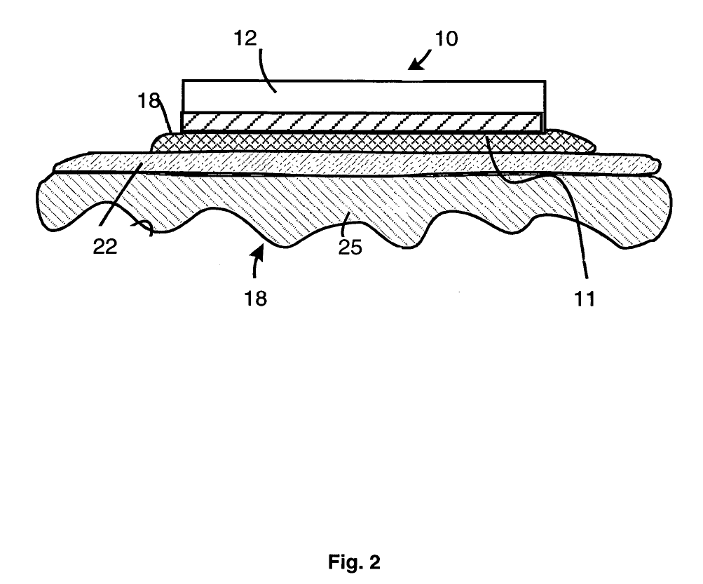

With continued reference to FIG. 1, FIG. 2 depicts a cross-sectional view of the device 10 when used on skin as the membrane 18. Here, the stratum corneum 22 is ablated by the abrasive 15 on the inferior surface 11 of the actuator 12, which is caused to rub back-and-forth due to the high frequency vibratory motion of the actuator 12. For the purpose of enhancing transdermal drug delivery, the depth of ablation does not extend any deeper than the epidermis 25.

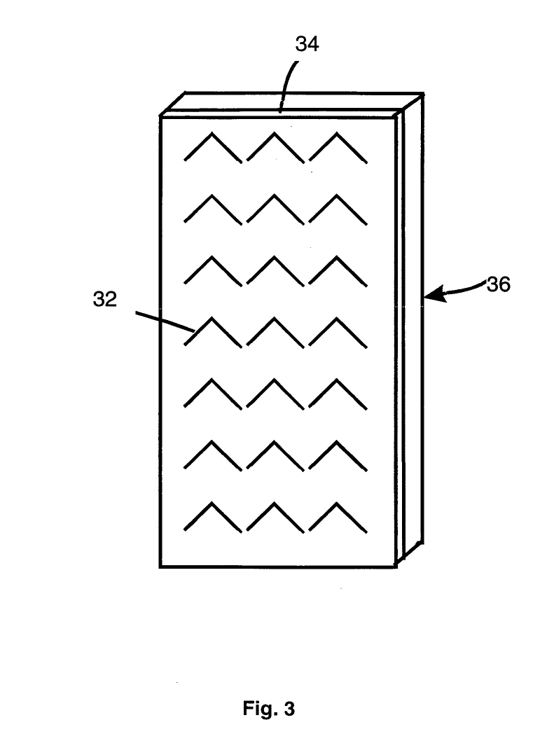

FIG. 3 depicts an embodiment of a modified actuator 36 that is placed against the membrane (not shown) to be treated. An array of chevrons 32 or ridges are disposed on the inferior surface 34 of the modified actuator 36 and extend beyond the inferior surface 34 of the modified actuator 36. With each stroke of the modified actuator 36, ablation takes place and the chevron structures 32 move the ablated material (not shown) to the side of the area being treated. The purpose of this is to remove material that does not take part in the ablative process. Alternatively, the textured actuator surface 34 itself can do the ablation without the need of applying an abrasive, such as shown in FIG. 1, between the actuator 36 and the membrane to be treated (not shown). Other examples of textured actuator surfaces are possible, e.g., random structures such as on sandpaper or microneedles.

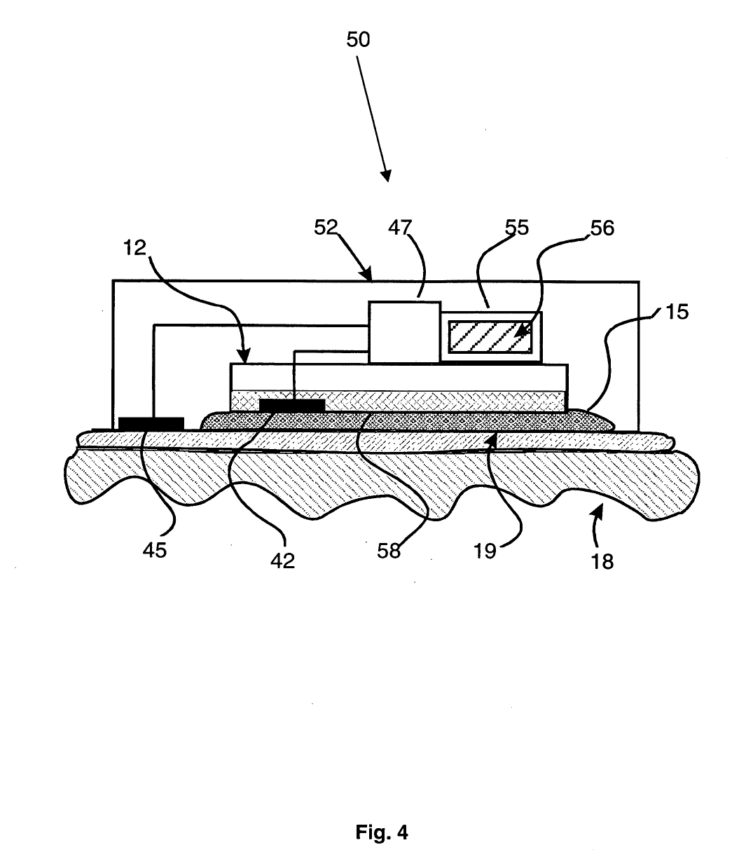

FIG. 4 depicts an alternate embodiment of the device 50. A piezoelectric actuator 12 functioning as a vibrating probe is associated with at least one electrode 42 that is in electrical contact with the ablation site 19 of the membrane 18. An abrasive 15 may be applied on the surface of the membrane 18. Optionally, the abrasive 15 may be fluidized such that a fluid interface is formed that improves the flow of charges between the surface of the electrode 42 and the ablation site 19. A second electrode 45 may be located distally from the first electrode 42 such that the membrane 18 forms a bridge between the electrodes 42,45 which may be composed of similar or different materials.

A microprocessor (not shown) present in a controller 47 generates a current across the electrodes 42, 45. The controller 47 detects changes in the condition of the treatment site and, according to an algorithm, sends a signal to continue or to cease the delivery of energy until a certain predetermined condition of the treatment site 20 is reached. A patch 52 containing the system contains a substance 56 held in a reservoir 55 to be delivered to the target site 20. In one form of the device a permeable membrane 58 modulates the release of the substance 56 to the treated site 20.

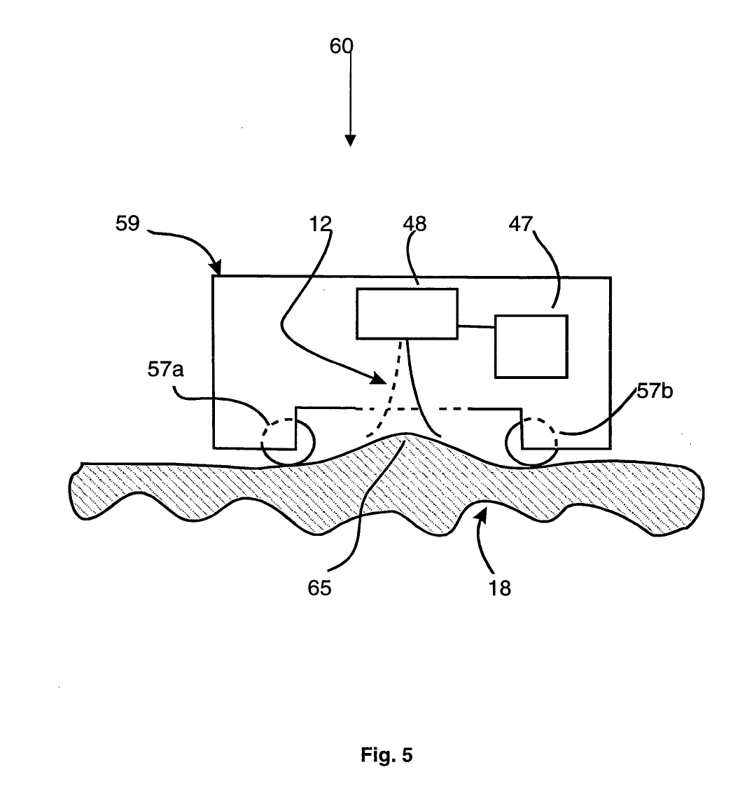

FIG. 5 depicts an alternate embodiment of the device 60. A piezoelectric actuator 12 is configured to vibrate in a bending mode by a power supply 48 and controller 47. The actuator and control electronics are contained within a housing 59 to which is attached cylindrical wheels 57a,b which allow the housing to be moved over the surface of the membrane 18. When downward pressure is applied to the housing 59, the membrane 18 extends upwards into the housing 59 at a position 65 whereupon the vibrating actuator 10 can come into contact with the membrane 18 and cause ablation.

The following examples are given for the purpose of illustrating various embodiments of the invention and are not meant to limit the present invention in any fashion.

### EXAMPLE 1

Ablation of Stratum Corneum using Micron Size Particles

Optimal particle size for removal of thin tissue layers was determined using a rotational surface applicator device with carborundum or aluminum oxide particles applied to the skin at various rotational frequencies. One adult male volunteer applied pressure at various rotational frequencies in order to determine the efficiency of removing the stratum corneum with regard to frequency and particle size. Frequency of the grinding device varied between 100 and 1000 Hz.

Fixed particle sizes below about 30 microns were inefficient, producing instead only minor abrasion or polishing. When particle size was increased to around 60-74 microns, the stratum corneum was removed efficiently and in a controlled manner as evidenced microscopically. Particle sizes of 100 micron or more produced excessive ablation and were difficult to control. Loose particles of aluminum oxide were found to be less efficient at removal of the outer layer of skin, and larger particle sizes were required, i.e., greater than 120 microns. Significant discomfort was generated during the less than 3 second treatments, possibly as a result of the lack of lubrication due to relatively low speed of the device, resulting in the generation of heat.

### EXAMPLE 2

Ablation of Stratum Corneum using a Piezoelectric Actuator

The effect of improved lubrication in the treatment area was studied through the use of a high frequency device which was anticipated to reduce the generation of heat. In this study, a piezoelectric actuator was used to apply a reciprocating force in a single direction in the plane of the skin surface, using carborundum or aluminum oxide particles, in an attempt to remove the outer skin layer. Multiple volunteers applied between 0.1 and 4 pounds of force to the area. The displacement of the actuator was between 50 and 250 microns, with a frequency set at 20 kHz. Efficiency of removing the stratum corneum with regard to frequency and particle size was evaluated.

In these experiments, fixed particle sizes below about 30 microns were inefficient, producing minor abrasion, or polishing. When particle size was increased to around 60-74 microns, stratum corneum was removed efficiently and in a controlled manner as evidenced microscopically. Particle sizes of 100 micron or more produced excessive shearing and were difficult to control. Loose particles of aluminum oxide were found to be less efficient at removal of the outer layer of skin, and larger particle sizes were required, i.e., greater than 120 microns. Each pulse of approximately 0.1 to 0.3 seconds removed a thin layer of the stratum corneum as evidenced microscopically and by the generation of a fine dust. Multiple pulses, i.e., between 5 and 20, resulted in complete removal of the stratum corneum and some of the epidermal layer. In some cases, bleeding was observed, however no pain or discomfort was noted.

### EXAMPLE 3

Dermal Resurfacing using Micron Size Particles

A rotational surface applicator device or planar piezoelectric actuator was used to apply an ablative force parallel to the skin surface using carborundum or aluminum oxide particles to remove the outer skin layer. Several volunteers applied force while testing various frequencies in order to determine the efficiency of polishing the skin with regard to frequency and particle size. Frequency of oscillatory motion of the devices was varied between 100 and 20,000 Hz. In these experiments, fixed particle sizes below about 30 microns were effective in producing minor abrasion, or polishing. Larger particle sizes, up to an exceeding 100 microns, produced significant dermabrasion as well.

### EXAMPLE 4

Delivery of a Topical Anesthetic Through Micro-Ablated Stratum Corneum

A piezoelectric actuator was fitted with 60 micron aluminum oxide particles fixed to its surface so as to provide an approximate 1 cm² treatment area. Ten to fifteen pulses of less than one second duration were applied with force of less than 1 pound to the area and frequency of 20 kHz. Significant ablation was documented by the appearance of fine white powder and redness or edema. A solution of 4% lidocaine was applied to the area and incubated for five minutes. The excess lidocaine was wiped off, and a series of probes was made in and around the area of treatment using a 20 gauge needle or by pinching. In these studies, it was determined that significant anesthesia was obtained through the treatment as evidenced by the lack of sensation within and in close proximity to the treatment site.

### EXAMPLE 5

Microdissection of Tissue

The efficiency of cutting tissue at high frequencies using a piezoelectric actuator fitted with carborundum or aluminum oxide particles where the longitudinal displacement of the actuator was held at a variety of angles relative to the surface to be cut was examined. The displacement of the actuator was between 50 and 250 microns, with a frequency set at 20 kHz. Forces of from 1 to 5 pounds were applied to excised, depilliated sheep skin. Efficiency of removing tissue with regard to frequency and particle size was evaluated. In these studies, each pulse of approximately 0.1 to 0.3 seconds cut into the tissue. When additional pressure was applied, and the angle increased, cutting of the tissue was possible.

Any patents or publications mentioned in this specification are indicative of the levels of those skilled in the art to which the invention pertains. These patents and publications are herein incorporated by reference to the same extent as if it was indicated that each publication was incorporated specifically and individually by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. A device for treating a tissue in an individual comprising:
an applicator,
a means to drive said applicator, and
an abrasive material.

2. The device of claim 1, further comprising a housing means.

3. The device of claim 1, wherein said tissue is altered or at least a portion of said tissue is ablated.

4. The device of claim 1, wherein said tissue is membranous or non-membranous.

5. The device of claim 4, wherein said membranous tissue is the stratum corneum.

6. The device of claim 5, wherein said non-membranous tissue is bone.

7. The device of claim 1, wherein said applicator comprises a rough-textured surface disposed adjacent said tissue or an actuator in contact with said tissue.

8. The device of claim 1, wherein said driving means is a piezoelectric material, a solenoid, a pressurized gas, an explosive discharge, a voice-coil, an electro- or magneto-responsive material, or an electro- or magneto-rheologic material, or a shape-memory alloy or polymer.

9. The device of claim 8, wherein said electro- or magneto-responsive material is polypyrrol.

10. The device of claim 8, wherein said electro-rheologic material is metallic filings dispersed in a viscous fluid.

11. The device of claim 8, wherein said magneto-rheologic material is magnetic filings dispersed in a viscous fluid.

12. The device of claim 8, wherein said shape-memory alloy is Nitonol.

13. The device of claim 8, wherein said driving means further comprises an electrophoretic means, mechanical pressure, osmotic pressure, hydrostatic pressure or a diffusion gradient.

14. The device of claim 1, wherein said abrasive material is biologically inert particles.

15. The device of claim 14, wherein said abrasive has a particle size of about 30 microns to about 120 microns.

16. The device of claim 15, wherein said abrasive has a particle size of about 50 microns to about 90 microns.

17. The device of claim 14, wherein said abrasive is diamond, aluminum oxide, carborundum, or ice.

18. The device of claim 1, wherein said abrasive further comprises a lubricant.

19. The device of claim 18, wherein said lubricant is water, a hydrogel, a lipid, aqueous carbohydrate, petrolatum, or glycerol or a combination thereof.

20. The device of claim 1, further comprising a means to deliver a pharmaceutical.

21. The device of claim 20, wherein said pharmaceutical is an anesthetic, nitroglycerin, an anti-nauseant, an antibiotic, a hormone, a steroidal antinflammatory agent, a non-steroid antiinflammatory agent, a chemotherapeutic agent, an anti-cancer agent, an immunogen, an anti-viral agent or an anti-fungal agent, or a diagnostic material.

22. The device of claim 21, wherein said antibiotic is tetracycline, streptomycin, sulfa drugs, kanamycin, neomycin, penicillin, or chloramphenicol.

23. The device of claim 21, wherein said hormone is parathyroid hormone, growth hormone, gonadotropins, insulin, ACTH, somatostatin, prolactin, placental lactogen, melanocyte stimulating hormone, thyrotropin, parathyroid hormone, calcitonin, enkephalin, or angiotensin.

24. The device of claim 21, wherein said anesthetic is lidocaine, bupivocaine, tetracaine, morphine, or fentanyl.

25. The device of claim 21, wherein said immunogen is a vaccine.

26. The device of claim 20, wherein said delivery means is said abrasive, wherein said abrasive is said pharmaceutical or said abrasive further comprises a lubricant containing said pharmaceutical.

27. The device of claim 26, wherein said pharmaceutical is a crystallized pharmaceutical or a powdered pharmaceutical

28. The device of claim 27, wherein said crystals are frozen.

29. The device of claim 20, wherein said delivery means comprises:
a reservoir containing said pharmaceutical, and
a permeable membrane through which said pharmaceutical is controllably released.

30. The device of claim 1, further comprising a collection means to collect ablated tissue or a biomolecule after treating said tissue at a site of interest.

31. The device of claim 30, wherein said collection means is a container operably connected to said device or an absorptive medium.

32. The device of claim 31, wherein said absorptive medium is activated carbon, a dehydrated hydrogel or cotton.

33. The device of claim 1, wherein said device is contained within a patch or said device is positioned on a probe, said probe insertable into a body cavity.

34. The device of claim 1, further comprising a control means to monitor feedback about an electrical property of said tissue, said control means comprising:
at least one first active electrode in electrical contact at a site of interest on said tissue;
a second return electrode in electrical contact distal to said first electrode at the site of interest;
an optional electrically conductive fluid interface between said first and second electrodes and the site of interest on said tissue; and
a controller to monitor an electrical current between said first electrode and said second electrode, said controller further comprising a microprocessor.

35. The device of claim 34, wherein said first electrode(s) and said second electrode and an electrolyte in body fluid in said tissue comprise a galvanic cell.

36. The device of claim 34, wherein said property is electrical impedence, electrical conductance, hydration, pH, or an endogenous electrical signal.

37. The device of claim 36, wherein said endogenous electrical signal is generated by a heartbeat or by brain activity of the individual.

38. A method to control the permeability of a tissue in an individual comprising the steps of:
contacting a site of interest on said tissue with the device of claim 34;
treating said tissue to ablate or alter said tissue at the site of interest;
monitoring an electrical property of said tissue at the site of interest;
applying an algorithm to evaluate said electrical property;
comparing the value obtained for said electrical property to a predetermined value wherein said values correlate to the permeability of said tissue; and
determining if said obtained value is at least equal to said predetermined value; and
signaling said device via said controller to continue said ablating or said altering if said obtained value does not at least equal said predetermined value thereby controlling the permeability of said tissue at the site of interest.

39. The method of claim 38 further comprising the step of delivering a pharmaceutical to the site of interest wherein said pharmaceutical is delivered during said monitoring step or subsequent to reaching said predetermined value of said physical property.

40. The method of claim 38 further comprising the step of collecting a biomolecule through said altered or ablated tissue when said predetermined value of permeability is reached.

41. The method of claim 38, wherein said predetermined value of said physical property is a known value or is obtained prior to treating said tissue.

42. The method of claim 41, wherein said predetermined value of said physical property is obtained from the same individual or within a group of individuals.

43. The device of claim 1, further comprising a control means to monitor feedback about an optical property of said tissue, said control means comprising:
at least one source of radiant energy directed at a site of interest on said tissue;
a light detector having optics with which to image said tissue thereon; and
a controller to monitor the radiant energy source and the light detector and to analyze data received from the light detector, said controller further comprising a microprocessor.

44. The device of claim 43, wherein said optical property is fluorescence or reflectance.

45. A method to control the permeability of a tissue in an individual comprising the steps of:
contacting a site of interest on said tissue with the device of claim 43;
treating said tissue to ablate or alter said tissue at the site of interest;
monitoring an optical property of said tissue at the site of interest;
applying an algorithm to evaluate said optical property;
comparing the value obtained for said optical property to a predetermined value wherein said values correlate to the permeability of said tissue; and
determining if said obtained value is at least equal to said predetermined value; and
signaling said device via said controller to continue said ablating or said altering if said obtained value does not at least equal said predetermined value thereby controlling the permeability of said tissue at the site of interest.

46. The method of claim 45 further comprising the step of delivering a pharmaceutical to the site of interest wherein said pharmaceutical is delivered during said monitoring step or subsequent to reaching said predetermined value of said physical property.

47. The method of claim 45 further comprising the step of collecting a biomolecule through said altered or ablated tissue when said predetermined value of permeability is reached.

48. The method of claim 45, wherein said predetermined value of said physical property is a known value or is obtained prior to treating said tissue.

49. The method of claim 48, wherein said predetermined value of said physical property is obtained from the same individual or within a group of individuals.

50. The device of claim 1, further comprising a control means to monitor feedback about a thermal property of said tissue, said control means comprising:
at least one source of infrared energy directed at a site of interest on said tissue;
an infrared detector having optics with which to measure infrared emission from said tissue thereon; and
a controller to monitor the infrared energy source and the infrared detector and to analyze data received from the light detector, said controller further comprising a microprocessor.

51. The device of claim 50, wherein said thermal property is thermal diffusivity and thermal conductivity.

52. A method to control the permeability of a tissue in an individual comprising the steps of:
contacting a site of interest on said tissue with the device of claim 50;
treating said tissue to ablate or alter said tissue at the site of interest;
monitoring a thermal property of said tissue at the site of interest;
applying an algorithm to evaluate said thermal property;
comparing the value obtained for said thermal property to a predetermined value wherein said values correlate to the permeability of said tissue; and
determining if said obtained value is at least equal to said predetermined value; and
signaling said device via said controller to continue said ablating or said altering if said obtained value does not at least equal said predetermined value thereby controlling the permeability of said tissue at the site of interest.

53. The method of claim 52 further comprising the step of delivering a pharmaceutical to the site of interest wherein said pharmaceutical is delivered during said monitoring step or subsequent to reaching said predetermined value of said physical property.

54. The method of claim 52 further comprising the step of collecting a biomolecule through said altered or ablated tissue when said predetermined value of permeability is reached.

55. The method of claim 52, wherein said predetermined value of said physical property is a known value or is obtained prior to treating said tissue.

56. The method of claim 55, wherein said predetermined value of said physical property is obtained from the same individual or within a group of individuals.

57. A method of treating a tissue in an individual comprising the steps of:
contacting said tissue in the individual at a site of interest with the device of claim 1; and
altering or ablating said tissue or a combination thereof at said site of interest with said device.

58. The method of claim 57 further comprising the step of delivering a pharmaceutical to said site of interest wherein said pharmaceutical is delivered simultaneously during said altering step or subsequent to said altering step.

59. A method for collecting a biomolecule from a tissue in an individual comprising the steps of:
contacting said tissue in the individual at a site of interest with the device of claim 1;
altering or ablating said tissue at the site of interest; and
collecting said biomolecule through said altered or ablated tissue at the site of interest wherein said biomolecule is collected in a container operably connected to said device.

60. A device for ablating tissue of an individual comprising:
an applicator,
a transducer to drive said applicator, and
an abrasive material comprised of particles of aluminum oxide, said particles having a particle size of about 30 microns to about 120 microns.

61. The device of claim 60, further comprising a lubricant of glycerol and water.

62. The device of claim 61, wherein said lubricant is electrically conductive.

63. A method of ablating tissue from an individual comprising the steps of:
contacting the tissue in the individual at a site of interest with the device of claim 60; and
ablating the tissue at the site of interest.

64. The method of claim 63 further comprising the step of delivering a pharmaceutical to the site of interest wherein said pharmaceutical is delivered simultaneously during said ablating step or subsequent to said ablating step.

65. A method for collecting a biomolecule from a tissue in an individual comprising the steps of:
contacting the tissue of the individual at a site of interest with the device of claim 60;
ablating said tissue at the site of interest; and
collecting said biomolecule from said tissue through said ablated tissue at the site of interest wherein said biomolecule is collected in a container operably connected to said device.

66. A device for ablating tissue of an individual comprising:
an actuator,
a transducer to drive said actuator;
a controller to control said transducer; and
a housing means further comprising two wheels rotatably attached thereto.

67. The device of claim 66, wherein said actuator is a piezoelectric actuator.

68. The device of claim 66, wherein said tissue is stratum corneum.

69. A method of ablating tissue from an individual comprising the steps of:
contacting said tissue in the individual at a site of interest with the device of claim 66;
applying downward pressure on the device to upwardly direct said tissue at the site of interest into said housing, said tissue in contact with the actuator; and
ablating said tissue at the site of interest via said actuator.
